(19) United States
(12) Patent Application Publication
(10) Pub. No.: US 2020/0143594 A1
(43) Pub. Date: May 7, 2020

(54) AUGMENTED REALITY VOICE-CONTROLLED SURGICAL SYSTEM

(71) Applicants: Tanmay Girish Lal, Moon Twp, PA (US); Robert Milliken, JR., Bethel Park, PA (US)

(72) Inventors: Tanmay Girish Lal, Moon Twp, PA (US); Robert Milliken, JR., Bethel Park, PA (US)

(21) Appl. No.: 16/381,439

(22) Filed: Apr. 11, 2019

Related U.S. Application Data
(60) Provisional application No. 62/756,300, filed on Nov. 6, 2018.

(51) Int. Cl. G06T 19/00; A61B 34/10; A61B 90/00; A61B 90/50

(52) U.S. Cl. CPC G06T 19/006; A61B 34/10; A61B 90/36; A61B 90/50; G02B 2027/0174; A61B 2090/374; A61B 2090/3762; A61B 2034/107; A61B 2090/365

(57) ABSTRACT

An augmented reality, voice-controlled surgical system is provided. The augmented reality, voice-controlled surgical system is adapted to superimpose a holographic image on a patient based on the patient's earlier-obtained radiological imagery. Augmented reality glasses are embodied in the present invention are electronically coupled to a systemic software application coupled to a database retrievably storing the radiological imagery. The hologram provides intra-operative detailed visualization in real time to the surgeon, wherein the system software selectively controls the holographic image in three-dimensions by voice commands.

### CROSS-REFERENCE TO RELATED APPLICATION

This application claims the benefit of priority of U.S. provisional application No. 62/756,300, filed 6 Nov. 2018, the contents of which are herein incorporated by reference.

### BACKGROUND OF THE INVENTION

The present invention relates to medical devices and, more particularly, to a voice controlled augmented reality system adapted to superimpose a hologram on a patient based on the patient's earlier-obtained radiological imagery.

Surgical procedures are inherently fraught with the risk, including, but not limited to, causing harm to surrounding vital structures, especially when attempting to address or remove deep-seated tissue damaged by injury or disease; for instance, during a surgical procedure to remove lesions in organs.

Currently there are no devices and or systems that can visualize the organ and the target tissue in real time through voice-activated commands that are effective in performing safe surgeries. The current radiological methods are old and do not provide intra-operative detailed visualization.

As can be seen, there is a need for augmented reality, voice-controlled system adapted to superimpose a hologram on a patient based on the patient's earlier-obtained radiological imagery, such as imagery from MRIs and CAT scans. The software-based system helps surgeons to localize deep seated lesions in organs so that they can be removed safely without damaging surrounding vital structures. The augmented realty software provides real time detail that is crucial for performing safe surgeries. The software is designed for augmented reality-based glasses that create holographic imagery of an organ and, for example, an underlying lesion during surgeries. Such holographic imagery is based on pre-existing radiological images accessible by the systemic software. Current methods do not provide the kind of detail that exists with the present invention, which affords surgeons intra-operative detailed visualization.

The software application is adapted to be a live reference for surgeons to selectively control through natural language commands. For example, the surgeon can verbally request animation of the slices/images for particular study during surgery, in real time. Without this invention, a surgeon, if their memory is unclear, would have to resort to leaving the operating table to reference the patient's information (tomographic, x-ray, et al.). This could impact the patient in longer anesthesia time, surgeon mistakes by feeling rushed, as well as add the possibility of contaminants. Because the software application is adapted for the surgeon to call up a 3D reference model of an organ and rotate it to get the best view possible, the present invention enhances recall of the surgeon where previously he/she would have to depend on memory before.

### SUMMARY OF THE INVENTION

In one aspect of the present invention, an augmented reality voice-controlled system for performing surgery on a patient includes the following: a database retrievably storing one or more radiological imagery of the patient; an augmented reality eyewear configured to display one or more holographic imagery in view of a wearer of said augmented reality eyewear, wherein the holographic imagery is based in part on said one or more radiological imagery; and systemic software operatively associated with each augmented reality eyewear so that each holographic imagery is selectively controlled by one or more voice commands.

In another aspect of the present invention, the augmented reality voice-controlled system for performing surgery on a patient includes the following: a database retrievably storing one or more radiological imagery of the patient; an augmented reality eyewear configured to display one or more holographic imagery in view of a wearer of said augmented reality eyewear, wherein the holographic imagery is based in part on said one or more radiological imagery; and systemic software operatively associated with each augmented reality eyewear so that each holographic imagery is selectively controlled by one or more voice commands, wherein the one or more voice commands include a attention system keyword, wherein the holographic imagery rotates along three orthogonal axis in three-dimensional space pursuant one or more voice commands, wherein the holographic imagery zooms in and out in three-dimensional space pursuant one or more voice commands, and wherein the holographic imagery comprises one or more surgical slices displayed in three-dimensional space pursuant one or more voice commands, wherein each surgical slice appears overlain on a portion of the patient associated with each surgical slice.

In yet another aspect of the present invention, method of remote learning and instruction for surgeons in a remote location could benefit from more-experienced surgeons in another location includes the following: providing the above-mentioned augmented reality voice-controlled system; providing the augmented reality eyewear in at least the remote location; and accessing the systemic software in at least the other location so that the one or more voice commands of each more-experienced surgeon selectively controls the holographic imagery displayed on the augmented reality eyewear.

These and other features, aspects and advantages of the present invention will become better understood with reference to the following drawings, description and claims.

### BRIEF DESCRIPTION OF THE DRAWINGS

FIG. 1 is a flow chart view of an exemplary embodiment of the present invention.

FIG. 2 is a schematic perspective view of an exemplary embodiment of the present invention shown in use;

FIG. 3 is a schematic elevation view of an exemplary embodiment of the present invention, shown in use; and

FIG. 4 is a schematic view of the process-oriented steps and components an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides augmented reality, voice-controlled surgical system adapted to superimpose a holographic image on a patient based on the patient's earlier-obtained radiological imagery. The augmented reality glasses embodied in the present invention are electronically coupled to the computer loaded with the systemic software application coupled to a database of said radiological imagery. The holographic images provides intra-operative detailed visualization in real time to the surgeon, wherein the systemic software selectively controls such images in three dimensions by voice commands.

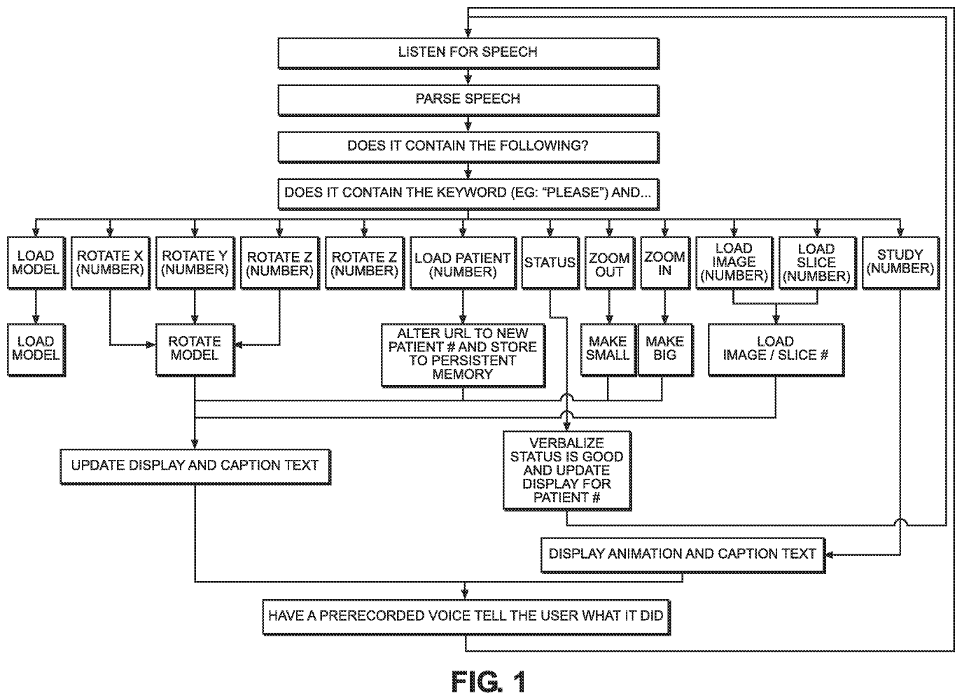

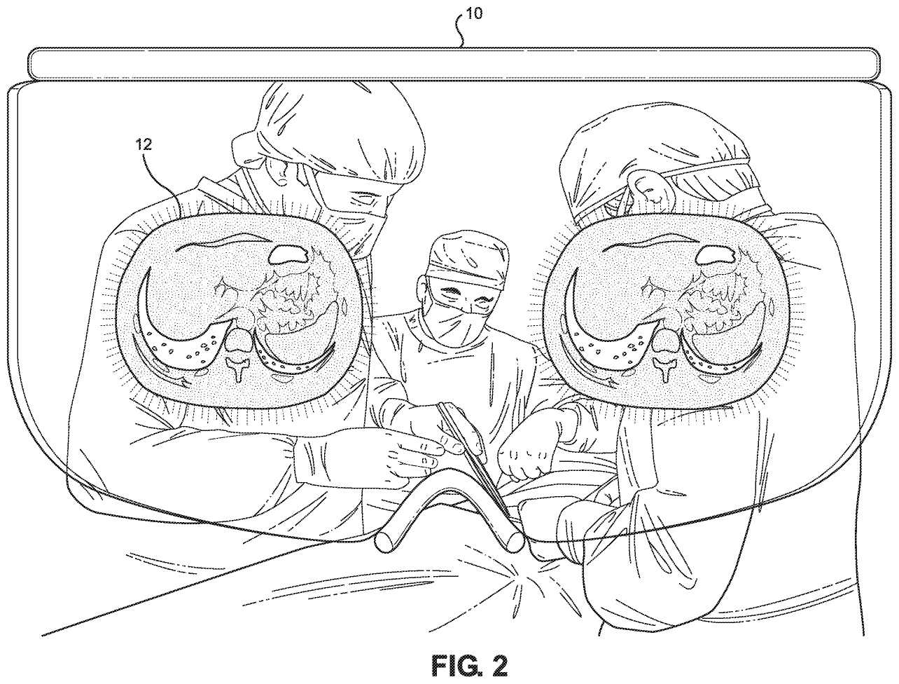

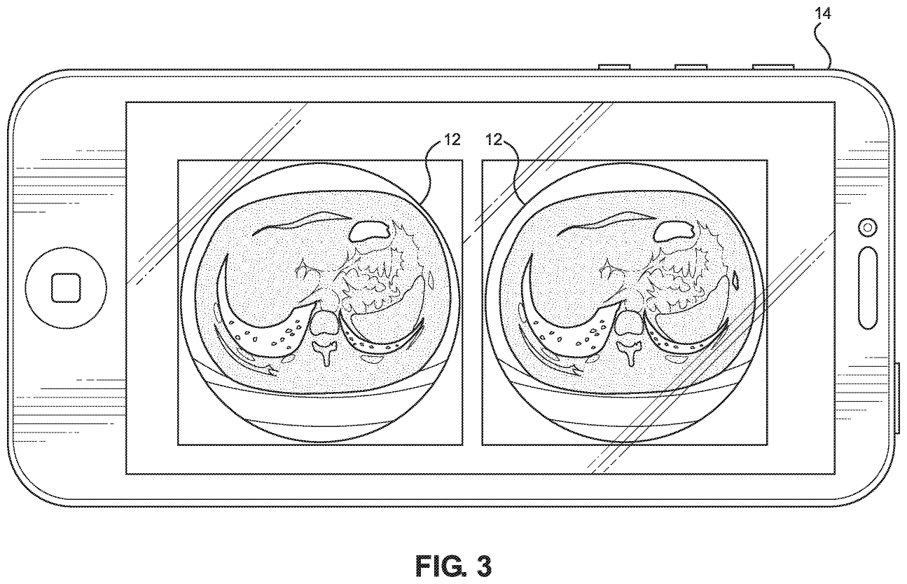

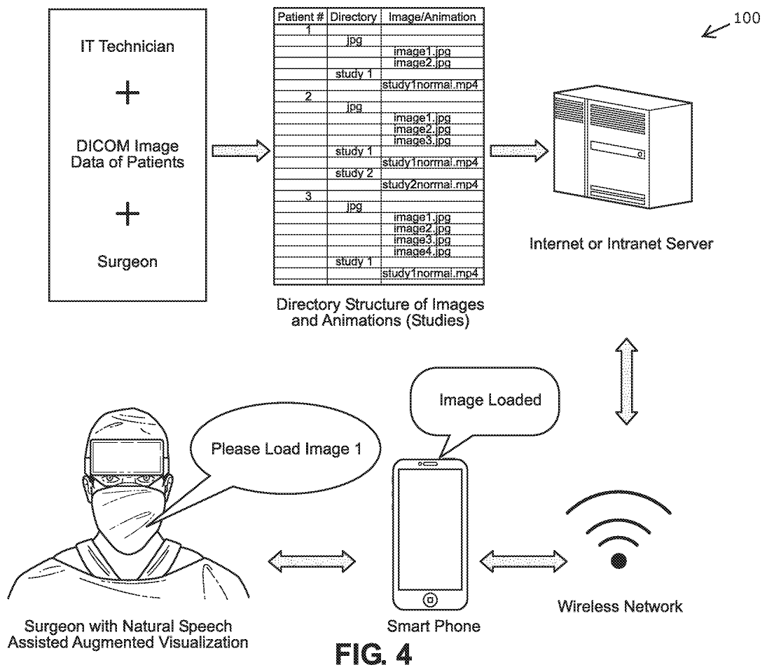

Referring to FIGS. 1 through 4, the augmented reality, voice-controlled surgical system 100 may include at least one computing device 14 with a user interface. The computing device 14 may include at least one processing unit coupled to a form of memory. The computing device 14 may include, but not limited to, a microprocessor, a server, a desktop, laptop, and smart device, such as, a tablet and smart phone. The computing device 14 includes a program product including a machine-readable program code for causing, when executed, the computing device 14 to perform steps. The program product may include software which may either be loaded onto the computing device 14 or accessed by the computing device 14. The loaded software may include an application on a smart device. The software may be accessed by the computing device 14 using a web browser. The computing device 14 may access the software via the web browser using the internet, extranet, intranet, host server, internet cloud and the like.

The voice-controlled surgical system 100 may embody augmented reality glasses 10 electronically coupled to the computing device 14 loaded with the software application. The computing device 14 may also electronically coupled to a database of radiological imagery, which may include imagery from MRIs, CAT scans or any imaging technologies such as X-ray radiography, medical ultrasonography endoscopy, elastography, tactile imaging, thermography, medical photography, positron emission tomography (PET), single-photon emission computed tomography (SPECT), or the like so as to represent holographic images 12 based on, at least in part, on said radiological imagery.

The software application is adapted to be a live reference for surgeons selectively controllable through natural language commands. The following are exemplary verbal commands. Note an “attention system” keyword may be used and can be replaced with something else. In the examples below, the keyword is “please”.

In certain embodiments, command words can be mixed (e.g., “load model please” is the same as “please load model”) except in the case of where a number is needed, then the word before it needs to describe what the number is used for (e.g., “please load image [number]”) is okay but “please load [number] image” is not). In addition, words can be added to the commands but will be ignored (e.g.: “please load image [number] right now” is the same as “please load image [number]”). Also note, the patient number is stored and if the application is closed and reopened that patient is remembered. The number can be a number from 1 through 4 or more. In certain embodiments, the numbers can only be done one at a time. The following are examples of voice-activated input that controls the surgical system 100—along with the system's 100 output:

“Please load model”—loads a 3D model.

“Please rotate x [number]”—rotates 3D model on the x-axis.

“Please rotate y [number]”—rotates 3D model on the y-axis.

“Please rotate z [number]”—rotates 3D model on the z-axis.

“Please load patient [number]”—Tells the application to switch to a different patient.

“Please study [number]”—Displays an animation of a predetermined range of slices and/or displays the animation in either zoomed out or zoomed in mode, depending on the current setting. The study number is displayed.

When these input commands are used, they call a server (computing device 14) that has these images/animations retrievably stored and display them to the end user, the surgeon. Anything that is black is transparent to the viewer, therefore the surgeon can view these images/animations while still seeing his/her environment. After a command is completed and the images/animations/models are displayed, the computer may be adapted to verbalize what it has just done (e.g., “Please load image (number)” has a verbal reply of “image loaded”), further informing the operator of the task at hand.

The integration of the ability to holographically display real time reference material in the operating theater in view of the surgeon's eyesight in the form of patient image slices, animations and models by voice command makes the present invention unique.

The advantages of this invention include convenience and ad hoc knowledge for the surgeon as well as increased safety for the patient. No longer will the surgeon rely strictly on memory or have to leave the operating table to recall from the possibility of hundreds of images from as many patients. The present invention has immediate access to and can selectively display in holographic imagery these associated images in the surgeon's point of view by voice-oriented input.

The proposed invention could create a hologram or image of the organ that the surgeon is working on and superimpose those images on the actual organ so as to create a realistic and accurate anatomical picture of the organ. This will help in the accurate dissection of the lesion within the organ, which often cannot be visualized as they are situated deep in the tissue. This will also ensure that the dissection and removal of the lesion does not damage surrounding vital structures.

The images available to the surgeon could be manipulated in three dimensions and retrieved in real time while the surgeon is performing an operation. This is in contrast to current radiological images that need the surgeon to move away from the operating room table or use additional devices (such as the ultrasound) in order to better visualize the proposed lesion that is being removed.

The proposed invention could be used for the purposes of education and training wherein the surgeon and the instructor, with the help of the same images on their individual AR headsets could effectively work together synergistically in order to perform surgeries safely.

The proposed invention could also be used for remote learning and instruction where surgeons in a remote location could benefit from the expertise of surgeons with more experience and training to guide them in surgeries where the patient cannot be transported to a tertiary care facility for their procedure.

A method of the using the present invention may include the following. The augmented reality surgical system disclosed above may be provided. The present invention is primarily useful to surgeons and surgical trainees/medical students during the planning and performance of difficult surgeries on vital organs such as the brain, liver etc. With the help of this software, under the selective control of voice command input, radiological images could be loaded on augmented-reality (AR) headsets/glasses 10 and be superimposed on the organ that is being operated upon. This will provide the surgeon with real time holographic three-dimensional images 12 of the organ and the lesion in 3D and color. This will help in accurate localization and removal of the lesion without damaging surrounding vital structures.

The radiological imagery may also be uploaded on a computer that then uses the software to create holographic imagery 12 that are then superimposed on the organ that is being operated upon via the augmented-reality glasses 10 worn by the surgeon. Non-surgical medical examination could also utilize the advantages of the present invention. Additionally, the present invention could be used in any field where complicated machines are being repaired or built.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. An augmented reality voice-controlled system for performing surgery on a patient, comprising:
a database retrievably storing one or more radiological imagery of the patient;
an augmented reality eyewear configured to display one or more holographic imagery in view of a wearer of said augmented reality eyewear, wherein the holographic imagery is based in part on said one or more radiological imagery; and
systemic software operatively associated with each augmented reality eyewear so that each holographic imagery is selectively controlled by one or more voice commands.

2. The augmented reality voice-controlled system for performing surgery on a patient of claim 1, wherein the one or more voice commands include a attention system keyword.

3. The augmented reality voice-controlled system for performing surgery on a patient of claim 1, wherein the holographic imagery rotates along three orthogonal axis in three-dimensional space pursuant one or more voice commands.

4. The augmented reality voice-controlled system for performing surgery on a patient of claim 1, wherein the holographic imagery zooms in and out in three-dimensional space pursuant one or more voice commands.

5. The augmented reality voice-controlled system for performing surgery on a patient of claim 1, wherein the holographic imagery comprises one or more surgical slices displayed in three-dimensional space pursuant one or more voice commands, wherein each surgical slice appears overlain on a portion of the patient associated with each surgical slice.

6. An augmented reality voice-controlled system for performing surgery on a patient, comprising:
a database retrievably storing one or more radiological imagery of the patient;
an augmented reality eyewear configured to display one or more holographic imagery in view of a wearer of said augmented reality eyewear, wherein the holographic imagery is based in part on said one or more radiological imagery; and
systemic software operatively associated with each augmented reality eyewear so that each holographic imagery is selectively controlled by one or more voice commands, wherein the one or more voice commands include a attention system keyword,
wherein the holographic imagery rotates along three orthogonal axis in three-dimensional space pursuant one or more voice commands, wherein the holographic imagery zooms in and out in three-dimensional space pursuant one or more voice commands, and wherein the holographic imagery comprises one or more surgical slices displayed in three-dimensional space pursuant one or more voice commands, wherein each surgical slice appears overlain on a portion of the patient associated with each surgical slice.

7. A method of remote learning and instruction for surgeons in a remote location could benefit from more-experienced surgeons in another location, comprising:
providing the augmented reality voice-controlled system of claim 6;
providing the augmented reality eyewear in at least the remote location; and
accessing the systemic software in at least the other location so that the one or more voice commands of each more-experienced surgeon selectively controls the holographic imagery displayed on the augmented reality eyewear.
